(19) United States
(12) Patent Application Publication
(10) Pub. No.: US 2005/0222269 A1
(43) Pub. Date: Oct. 6, 2005

(54) Use of deprenyl compounds to treat viral infections and reduce tissue damage associated therewith

(76) Inventors: William G. Tatton, Fort MacLeod (CA); Katherine L.B. Borden, New Rochelle, NY (US); Maria Salvato, Madison, WI (US)

Correspondence Address: LAHIVE & COCKFIELD, LLP., 28 STATE STREET, BOSTON, MA, 02109, US

(21) Appl. No.: 11/048,116

(22) Filed: Jan. 31, 2005

Related U.S. Application Data
(63) Continuation of application No. 09/249,350, filed on Feb. 11, 1999, now abandoned.
(60) Provisional application No. 60/074,449, filed on Feb. 12, 1998.

(51) Int. Cl. A61K 31/137; A61K 31/445; A61K 31/40

(52) U.S. Cl. 514/649; 514/408; 514/317

(57) ABSTRACT

Methods for treating viral infections are disclosed. The methods of the invention are useful for inhibiting viral infection in a subject and for preventing reducing tissue damage associated with viral infections. The method can include the step of administering to a subject in need thereof a therapeutically effective amount of a deprenyl compound, such that treatment of a viral infection occurs.

### RELATED APPLICATIONS

This application is a continuation of U.S. application Ser. No. 09/249,350, filed Feb. 11, 1999; which claims priority to U.S. Provisional Application No. 60/074,449, filed Feb. 12, 1998. The entire contents of both of these applications are hereby incorporated in their entirety by reference.

### BACKGROUND OF THE INVENTION

Viral infections remain one of the most challenging obstacles in modern medicine. Viruses are responsible for a variety of widespread, serious human diseases, including acquired immune deficiency syndrome (AIDS), which is caused by the human immunodeficiency virus (HIV). Other viruses which cause human disease include, e.g., Herpes Simplex-1 virus, hepatitis A virus, Epstein-Barr virus, SV-40 virus, cytomeglavirus and adenovirus-5.

Treatment of viral infection has historically been difficult. In many cases, such as the common cold, only limited symptomatic relief has been generally available. Although many antibiotics are now available for effective treatment of bacterial infections, until recently very few pharmaceutical agents had any significant effect on viral infections. Among the few pharmaceutical agents capable of inhibiting viral replication are nucleoside analogs such as zidovudine (AZT) and didenosine. Unfortunately, some viruses are capable of developing resistance to these drugs.

More recently, inhibitors of viral protease enzymes (such as HIV protease) have been found useful for the treatment of certain viral infections, such as HIV infection. However, these viral protease inhibitors have generally been designed to inhibit the action of a particular, virus-specific enzyme. The process of designing and synthesizing specific drugs is time-consuming and expensive, often requiring a detailed knowledge of the viral life cycle and of the structure of the specific enzymes implicated in viral replication. Moreover, the resulting agents (e.g., protease inhibitors) usually do not have a broad spectrum of activity against viruses other than the particular target virus. Thus, a different compound would need to be designed for each virus to be treated.

There is therefore a need for effective, general therapies which can target the viral replication system in a way that prevents or decreases viral replication in vivo.

Deprenyl (also known as selegiline or R-(−)—N,α-Dimethyl-N-2-propynyl phenethylamine) was first used as an adjunct to conventional drug therapy of Parkinson's disease (PD) in Europe over a decade ago on the basis that it is a selective monoamine oxidase-B(MAO-B) inhibitor. It has been reported that deprenyl and related compounds are capable of rescuing damaged nerve cells. However, the use of deprenyl and related compounds to inhibit viral replication has not been reported.

### SUMMARY OF THE INVENTION

This invention relates to methods for treating viral infections. The methods of the invention are useful for inhibiting viral infection in a subject and for preventing reducing tissue damage associated with viral infections.

In one aspect, the invention provides a method of treating a viral infection. The method includes the step of administering to a subject in need thereof a therapeutically effective amount of a deprenyl compound, such that treatment of the viral infection occurs. In certain embodiments, the viral infection is caused by an RNA virus, such as HIV, Herpes Simplex-1 virus, hepatitis A virus, Epstein-Barr virus, SV-40 virus, cytomeglavirus and adenovirus-5. In preferred embodiments, the deprenyl compound can be represented by the formula: 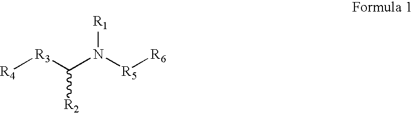
in which R₁ is hydrogen, alkyl, alkenyl, alkynyl, aralkyl, alkylcarbonyl, arylcarbonyl, alkoxycarbonyl, or aryloxycarbonyl; R₂ is hydrogen or alkyl; R₃ is a single bond, alkylene, or —(CH₂)ₙ—X—(CH₂)ₘ; in which X is O, S, or N-methyl; m is 1 or 2; and n is 0,1, or 2; R₄ is alkyl, alkenyl, alkynyl, heterocyclyl, aryl or aralkyl; R₅ is alkylene, alkenylene, alkynylene and alkoxylene; and R₆ is C₃-C₆ cycloalkyl or —C≡CH; or R₂ and R₄-R₃ are joined to form, together with the methine to which they are attached, a cyclic or polycyclic group; and pharmaceutically acceptable salts thereof. A particularly preferred deprenyl compound is (−)-desmethyldeprenyl. The deprenyl compound can be administered to the subject by any suitable route of administration, including transdermal administration, and may be administered in a pharmaceutically acceptable carrier. In certain embodiments, the subject is a human.

In another aspect, the invention provides a method of inhibiting replication of a virus in a virus-infected cell. The method includes the step of contacting the virus-infected cell with an effective amount of a deprenyl compound, such that the affinity of GAPDH for viral RNA is decreased and viral replication in the virus-infected cell is inhibited. The virus can be, for example, HIV, Herpes Simplex-1 virus, hepatitis A virus, Epstein-Barr virus, SV-40 virus, cytomeglavirus and adenovirus-5. The virus-infected cell can be a cell in cell culture. The deprenyl compound is preferably a compound which can be represented by Formula I (supra), and preferably is desmethyldeprenyl, more preferably (−)-desmethyldeprenyl.

In another aspect, the invention provides a method for decreasing the affinity of GAPDH for viral RNA. The method includes the step of contacting GAPDH with a deprenyl compound, such that the affinity of GAPDH for viral RNA is decreased. In certain embodiments, the deprenyl compound associates with GAPDH such that the conformation of GAPDH is altered. The deprenyl compound is preferably a compound which can be represented by Formula I (supra), and preferably is desmethyldeprenyl, more preferably (−)-desmethyldeprenyl.

In yet another aspect, the invention provides a method for inhibiting replication of a virus in a virus-infected cell, comprising inhibiting colocalization of GAPDH with PML such that replication of the virus in the virus-infected cell is inhibited. In certain embodiments, the colocalization of GAPDH with PML is inhibited by contacting GAPDH with a deprenyl compound, such as compound represented by Formula I (supra), for example, desmethyldeprenyl, more preferably (−)-desmethyldeprenyl.

In still another aspect, the invention provides a method for inhibiting tissue damage due to viral infection, comprising administering to a subject in need thereof an effective amount of a deprenyl compound such that prevention of tissue damage due to viral infection occurs. The virus can be, for example, HIV, Herpes Simplex-1 virus, hepatitis A virus, Epstein-Barr virus, SV-40 virus, cytomeglavirus and adenovirus-5. The deprenyl compound is preferably a compound which can be represented by Formula I (supra), and preferably is desmethyldeprenyl, more preferably (−)-desmethyldeprenyl. In certain preferred embodiments, the patient is a human.

The invention further provides a method of treating viral infections by administering an effective amount of a deprenyl compound to a human subject to treat viral infections.

The invention also provides a method of treating viral infection, comprising inhibiting GAPDH from contacting viral RNA such that treatment of viral infection occurs.

The invention also provides a method of inhibiting viral replication, comprising administering a deprenyl compound to a subject such that prevention of viral replication occurs.

The invention further provides a method of inhibiting viral replication, comprising binding a deprenyl compound to GAPDH such that prevention of viral replication occurs.

The invention also provides a method of inhibiting viral replication, comprising inhibiting GAPDH from contacting viral RNA such that prevention of viral replication occurs.

The invention also provides a method for inhibiting viral replication, comprising inhibiting colocalization of a deprenyl-GAPDH complex with PML such that prevention of viral replication occurs.

The invention also provides a method for inhibiting tissue damage due to viral infection, comprising administering a deprenyl compound to a subject such that prevention of tissue damage due to viral infection occurs.

The invention also provides a method for inhibiting tissue damage due to viral infection, comprising binding a deprenyl compound to GAPDH to form a complex such that prevention of tissue damage due to viral infection occurs.

The invention also provides a method of inhibiting tissue damage due to viral infection, comprising inhibiting GAPDH from contacting viral RNA such that prevention of tissue damage due to viral infection occurs.

### DETAILED DESCRIPTION OF THE INVENTION

The present invention is related to methods for treating viral infections and the tissue damage that is associated therewith, e.g., by administering a deprenyl compound to a patient. The invention is based, at least in part, on the discovery that deprenyl or deprenyl compounds can bind to GAPDH and alter or inhibit viral replication and/or translation of viral nucleic acids.

The terms “patient or subject”, as used herein, refer to a warm-blooded animal having a viral infection. In preferred embodiments, the patient is a mammal, including humans and non-human mammals, such as dogs, cats, pigs, cows, sheep, goats, rats and mice. In a particularly preferred embodiment, the patient is a human.

The terms “viral infection” or “virus infection”, as used herein, refer to the infection of a cell or a subject by a virus. Thus, a “viral infection” includes infection of a cell, or a symptomatic or asymptomatic infection of a subject by a virus.

The term “inhibiting viral infection” as used herein, refers to decreasing the ability of a virus to infect a cell or a subject, or decreasing, inhibiting, slowing, ameliorating, or reversing the course of a viral infection in a cell or a subject. Thus, for example, “inhibiting viral infection” includes, e.g., preventing a virus from infecting a cell, preventing or decreasing viral replication in an infected cell or subject, and the like. Similarly, “treating” a viral infection refers to decreasing, inhibiting, slowing, ameliorating, or reversing the course of a viral infection, or symptoms thereof, in a cell or subject.

The term “tissue damage”, as used herein, refers to damage to cells or tissues, e.g., cell death, impairment of cell or tissue structure or function, and the like.

Methods for Treating Viral Infection

As discussed in more detail infra, it has now been found that deprenyl compounds are capable of preventing or inhibiting viral infection and/or viral replication. Without wishing to be bound by any theory, it is believed that deprenyl compounds interact (e.g., bind) with glyceraldehyde-3-phosphate dehydrogenase (GAPDH), and we believe that the binding with GAPDH is responsible, at least in part, for the effects of deprenyl compounds on viral infection and/or replication.

Thus, in one aspect, the invention provides a method for treating a viral infection. The method includes the step of administering to a subject in need thereof a therapeutically effective amount of a deprenyl compound, optionally in a pharmaceutically-acceptable carrier, such that treatment of a viral infection occurs.

In accordance with the present invention, a subject suffering from a viral infection can be treated by administering an effective amount of a deprenyl compound, such that the viral infection is treated. Thus, a deprenyl compound can be administered in an amount, and for a period of time, sufficient to treat a pre-existing viral infection, whether or not symptoms of the viral infection are evident in the patient. It will be understood that the amount of the deprenyl compound which is administered to the subject may vary according to such factors as the type and severity of the viral infection to be treated.

The present invention provides therapies for infection by a variety of viruses (preferably RNA viruses), including, but not limited to, diseases of humans caused by viruses such as the human immunodeficiency virus, influenza virus, RNA tumor virus, Herpes Simplex-1 virus, hepatitis A virus, Epstein-Barr virus, SV-40 virus, cytomegalovirus, adenovirus-5, and the like; and diseases of animals (such as domestic or experimental animals) including Boma virus and the like.

It will be appreciated that treatment of viral infections according to the present invention can include any of the following outcomes: cure of the viral infection, e.g., eradication of virus from the subject; slowing or reversal of the course of a viral infection, e.g., the rate of viral infection or the number or severity of symptoms associated with the viral infection; decrease in transmissibility of the viral infection from one subject to another; and the like. Thus, although the goal of treatment will generally be eradication of the viral infection, alleviation of symptoms can be a principal or subsidiary goal of treatment according to the invention. For example, the nervous system damage that accompanies AIDS is one of the particularly debilitating aspects of the disease and could be controlled by administration of a deprenyl compound (e.g., (−)-desmethyldeprenyl) to an AIDS patient.

In addition, an effective amount of a deprenyl compound can be administered to the subject as a prophylaxis against viral infection, i.e., to prevent, inhibit, slow, or otherwise decrease the likelihood of, viral infection in the patient. In this embodiment, the invention provides a broad-spectrum preventive therapy for decreasing the likelihood that a patient will contract a viral illness.

The deprenyl compound can be any compound which is structurally or functionally similar to deprenyl, as described in more detail infra. The deprenyl compound can be administered to the subject according to any effective route of administration, including the methods described hereinbelow. The deprenyl compound is preferably adminstered in a pharmaceutically acceptable carrier.

There are at least three mechanisms by which deprenyl compounds could alter viral pathogenesis (including, e.g., viral infection, replication, and damage to host cells):

First: GAPDH may play a direct role in the viral replication process by unwinding upstream regulatory regions of the viral RNA, thereby altering virus production. The binding of GAPDH to AU rich segments of RNA, which often are formed into hairpin loops, has been described; the RNA binding site of GAPDH is at the NAD binding pocket known as the Rossman fold (Nagy and Rigby, 1995). GAPDH has been shown to bind 5′ non translated RNA regions of the Hepatitis A virus (Schultz et al., 1996). It was observed that GAPDH destabilized RNA helices. Because of the capacity to destabilize RNA helices, GAPDH may effect internal ribosomal entry site (IRES) dependent translation and therefore the replication of picornaviruses, like Hepatitis A, which depend on the IRES for their replication. Thus, GAPDH may unwind the viral RNA making it more readily available to the host cell's translational machinery. As described in more detail below, it has now been discovered that deprenyl compounds can bind to GAPDH. It is believed that binding of deprenyl compounds to GAPDH can affect binding of GAPDH to viral RNA. By decreasing the affinity of GAPDH for viral nucleic acids, deprenyl compounds can decrease unwinding of viral nucleic acids, thereby reducing production of viral gene products.

We have now performed molecular modeling and confocal laser imaging studies which indicate that at least certain deprenyl compounds have the capacity to induce conformational changes in GAPDH and therefore may alter GAPDH binding to RNA and/or other proteins. Moreover, the binding of deprenyl compounds to GAPDH can promote the formation of dimeric GAPDH rather than tetrameric GAPDH, with resultant changes in the ability of GAPDH to bind to RNA rather than DNA (see Example 1, infra).

Second: GAPDH could affect viral replication through an interaction with promyelocytic leukemia (PML) protein. The present inventors have found that nuclear immunoreaction for GAPDH co-localizes with that for a subset of nuclear bodies associated with PML, a growth suppresser protein. Nuclear PML bodies are a target of several viruses since in some cases, viral replication sites form nearby to PML nuclear bodies. Alterations in PML bodies have been described for a number of double stranded DNA viruses including Herpes Simplex-1, Cytomegalovirus, Epstein Barr virus, adenovirus 5, Pappiloma virus and SV-40 (Doucas and al., 1996; Ishov and Maul, 1996; Kelly et al., 1995; Maul et al., 1993; Puvion-Dutilleul and al., 1995; Szekely, 1996; Day et al., 1998). PML bodies are also disrupted during infection with Lymphocytic Choriomeningitis virus (LCMV), a single stranded ambisense RNA virus from the arenavirus family. Results with another member of the arenaviridae, Lassa, indicate that the PML protein is targeted by this virus; this targeting may be a general feature of arenaviruses. Since PML and GAPDH co-localize to the same bodies, the virus may be targeting both GAPDH and PML. Thus, deprenyl compounds with the ability to alter the conformation, subcellular distribution and/or activity of GAPDH might directly or indirectly effect PML bodies, altering the ability of a virus to commandeer the host cell machinery that it requires for replication.

Third, alterations in the ability of a cell to apoptose can alter viral pathogenicity. The pathogenesis of some viruses is mediated not only through viral replication, but by immunopathological events. In particular, viral infections can cause cytotoxic T lymphocytes (CTL) to attack and kill cells by apoptosis. During LCMV infection this effect is responsible for causing the choriomeningitis observed in patients infected with this virus (reviewed by (Salvato and Rai, 1996)); the production of neuralgia in Lassa fever patients (reviewed by (Salvato and Rai, 1996)); and AIDS induced pathogenesis, in particular HIV induced encephalitis(reviewed by (Kalams and Walker, 1992)). Similar findings have been reported for Simian Immunodeficiency virus (SIV) (Kalams and Walker, 1992) and Boma disease, a naturally occurring neurological disorder in horses, sheep, and cattle (Morimoto and al., 1996; Sobbe and al., 1997). The capacity of the virus to induce apoptosis through an immune reaction forms the basis for the cytotoxic T lymphocyte (CTL) test. GAPDH has been linked to neuronal apoptosis (Ishitani et al., 1996b). Thus, drugs that affect CTL induced apoptosis through GAPDH and/or PML, can change the progression of tissue damage associated with infection. (−)-Deprenyl (Tatton et al., 1994), and (−)-desmethyldeprenyl, the primary metabolite of (−)-deprenyl, have been found to mediate changes in new protein synthesis and can inhibit apoptosis The present inventors also have found that several other (−)-deprenyl analogs can mediate anti-apoptosis. Differential display PCR has shown that N-acetyl-1-cysteine (NAC) induces almost identical changes in gene expression to (−)-deprenyl and (−)-desmethyldeprenyl. NAC reduces apoptosis ( ) by a mechanism that requires new protein synthesis, but NAC does not share the capacity of deprenyl compounds to reduce virus mediated CTL. As described in Example 1, infra, fluorescent labelling of (−)-deprenyl analogues allowed the identification of binding proteins for the analogues, one such protein being GAPDH. Thus, according to one preferred practice of the invention, a deprenyl compound is administered to a subject in an amount sufficient to inhibit viral replication in cells of the subject. For example, a deprenyl compound can be administered to the subject in an amount sufficient to inhibit an association between GAPDH and a viral nucleic acid, such that viral replication is inhibited, e.g., by preventing or decreasing unwinding of viral RNA and reducing or preventing production of viral gene products by a host cell. In this embodiment, a deprenyl compound is administered in an amount sufficient to bind to, or associate with, a fraction of intracellular GAPDH effective to significantly decrease the binding of GAPDH to viral RNA, such that the amount of viral gene products translated by the host cell is decreased. For example, a deprenyl compound can be administered to a subject in an amount sufficient to alter the aggregation of GAPDH monomers, e.g., to promote the conversion of tetrameric GAPDH to dimeric GAPDH, such that the relative affinity of GAPDH for both DNA and RNA is altered. Thus, the amount of deprenyl compound administered to the subject is preferably sufficient to promote the formation of dimeric GAPDH such that the affinity of GAPDH for viral RNA is decreased.

In another embodiment, a deprenyl compound can be administered to the subject in an amount sufficient to alter the intracellular distribution of GAPDH, such that viral replication is inhibited, e.g., by preventing co-localization of GAPDH with PML nuclear bodies. In this embodiment, the intracellular distribution of GAPDH is altered by binding of a deprenyl compound to a sufficient fraction of GAPDH to significantly alter the intracellular distribution of GAPDH and inhibit viral replication, e.g., by preventing the virus from effectively utilizing the host cell to produce new virus.

In still another embodiment, a deprenyl compound can be administered to the subject in an amount sufficient to prevent apoptosis of an infected cell or cells, e.g., by preventing CTL-mediated cell killing of virally-infected cells, such that cell death due to viral infection is decreased. In this embodiment, the amount of deprenyl compound administered to the subject is preferably sufficient to prevent apoptosis by preventing an interaction of CTLs with infected cells, or by preventing the association of CTLs with infected cells from resulting in death of the infected cells.

In another aspect, the invention provides a method of decreasing viral replication in a virus-infected cell, e.g., by decreasing the affinity of GAPDH for viral RNA. The method comprises contacting the virus-infected cell with an effective amount of a deprenyl compound, such that the affinity of GAPDH for viral RNA is decreased and viral replication in the virus-infected cell is inhibited, e.g., compared to viral replication in a control cell not treated with a deprenyl compound. The virus-infected cell can be a cell maintained in vitro, e.g., in cell culture, or the cell can be a cell of a subject.

In another aspect, the invention provides a method for decreasing the affinity of GAPDH for viral RNA. The method includes the step of contacting GAPDH with a deprenyl compound, such that the affinity of GAPDH for viral RNA is decreased. In preferred embodiments, the deprenyl compound associates with GAPDH such that the conformation of GAPDH is altered.

In another aspect, the invention provides a method for inhibiting replication of a virus in a virus-infected cell. The method includes the step of inhibiting colocalization of GAPDH with PML such that replication of the virus in the virus-infected cell is inhibited. In preferred embodiments, the colocalization of GAPDH with PML is inhibited by contacting GAPDH with a deprenyl compound.

In another aspect, the invention provides a method for inhibiting tissue damage due to viral infection. The method includes administering to a subject in need thereof an effective amount of a deprenyl compound such that prevention of tissue damage due to viral infection occurs.

I. Deprenyl Compounds

The language “deprenyl compound”, as used herein, includes deprenyl (N,α-dimethyl-N-2-propynylphenethylamine), compounds which are structurally similar to deprenyl, e.g., structural analogs, or derivatives thereof. Thus, in one embodiment, a deprenyl compound can be represented by the following general formula (Formula 1): 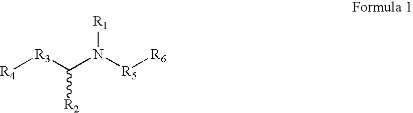
in which
R₁ is hydrogen, alkyl, alkenyl, alkynyl, aralkyl, alkylcarbonyl, arylcarbonyl, alkoxycarbonyl, or aryloxycarbonyl;
R₂ is hydrogen or alkyl;
R₃ is a single bond, alkylene, or —(CH₂)ₙ—X—(CH₂)ₘ;
in which X is O, S, or N-methyl; m is 1 or 2; and n is 0,1, or 2;
R₄ is alkyl, alkenyl, alkynyl, heterocyclyl, aryl or aralkyl; and
R₅ is alkylene, alkenylene, alkynylene and alkoxylene; and
R₆ is C₃-C₆ cycloalkyl or
   —C≡CH; or
R₂ and R₄-R₃ are joined to form, together with the methine to which they are attached, a cyclic or polycyclic group;
and pharmaceutically acceptable salts thereof. In certain embodiments: R₁ is a group that can be removed in vivo; R₁ is hydrogen; R₁ is alkyl; R₁ is methyl; R₂ is methyl; R₃ is methylene; wherein R₄ is aryl; R₄ is phenyl; R₅ is methylene; R₆ is
   —C≡CH

In certain preferred embodiments the deprenyl is represented by the following general formula (Formula 2): 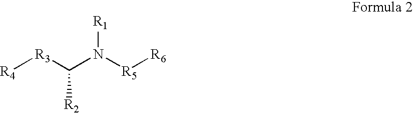
in which
R₁ is hydrogen, alkyl, alkenyl, alkynyl, aralkyl, alkylcarbonyl, arylcarbonyl, alkoxycarbonyl, or aryloxycarbonyl;
R₂ is alkyl;
R₃ is a single bond, alkylene, or —(CH₂)ₙ—X—(CH₂)ₘ;
   in which X is O, S, or N-methyl; m is 1 or 2; and n is 0,1, or 2;
R₄ is alkyl, alkenyl, alkynyl, heterocyclyl, aryl or aralkyl; and
R₅ is alkylene, alkenylene, alkynylene and alkoxylene; and
R₆ is C₃-C₆ cycloalkyl or
   —C≡CH; or
R₂ and R₄-R₃ are joined to form, together with the methine to which they are attached, a cyclic or polycyclic group;
and pharmaceutically acceptable salts thereof. In certain embodiments: R₁ is a group that can be removed in vivo; R₁ is hydrogen; R₁ is alkyl; R₁ is methyl; R₂ is methyl; R₃ is methylene; wherein R₄ is aryl; R₄ is phenyl; R₅ is methylene; R₆ is
   —C≡CH

In certain preferred embodiments, the deprenyl is represented by the following general formula (Formula 3): 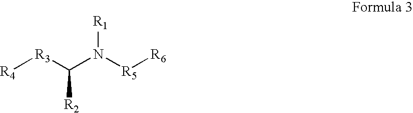
in which
R₁ is hydrogen, alkyl, alkenyl, alkynyl, aralkyl, alkylcarbonyl, arylcarbonyl, alkoxycarbonyl, or aryloxycarbonyl;
R₂ is alkyl;
R₃ is a single bond, alkylene, or —(CH₂)ₙ—X—(CH₂)ₘ;
in which X is O, S, or N-methyl; m is 1 or 2; and n is 0,1, or 2;
R₄ is alkyl, alkenyl, alkynyl, heterocyclyl, aryl or aralkyl; and
R₅ is alkylene, alkenylene, alkynylene and alkoxylene; and
R₆ is C₃-C₆ cycloalkyl or
   —C≡CH; or
R₂ and R₄-R₃ are joined to form, together with the methine to which they are attached, a cyclic or polycyclic group;
and pharmaceutically acceptable salts thereof. In certain embodiments: R₁ is a group that can be removed in vivo; R₁ is hydrogen; R₁ is alkyl; R₁ is methyl; R₂ is methyl; R₃ is methylene; wherein R₄ is aryl; R₄ is phenyl; R₅ is methylene; R₆ is
   —C≡CH
   In another preferred embodiments, R₆ is cyclopentyl.

In a preferred embodiment, the deprenyl compound is N-propargyl-1-aminoindan, more preferably (R)-N-propargyl-1-aminoindan (see, e.g., International Publication No. WO 95/11016, incorporated herein by reference). In another preferred embodiment, the deprenyl compound is a 1-aminoaliphatyl-dibenz[b,f]oxepine such as 10-(N-propargylamino)methyl dibenz[b,f]oxepine or 10-(N-methyl-N-propargylamino)methyl dibenz[b,f]oxepine (see, e.g., m European patent Publication No. EP 726265, incorporated herein by reference).

In certain preferred embodiments, the deprenyl compound is represented by the following general formula (Formula 4): 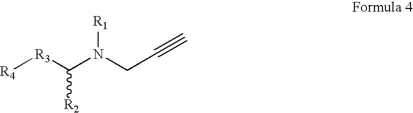
in which
R₁ is hydrogen, alkyl, alkenyl, alkynyl, aralkyl, alkylcarbonyl, arylcarbonyl, alkoxycarbonyl, or aryloxycarbonyl;
R₂ is hydrogen or alkyl;
R₃ is a bond or methylene; and

R₄ is aryl or aralkyl; or
R₂ and R₄-R₃ are joined to form, together with the methine to which they are attached, a cyclic or polycyclic group;
and pharmaceutically acceptable salts thereof.

In certain preferred embodiments, the deprenyl is represented by the following general formula (Formula 5): 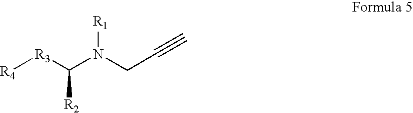
in which
R₁ is hydrogen, alkyl, alkenyl, alkynyl, aralkyl, alkylcarbonyl, arylcarbonyl, alkoxycarbonyl, or aryloxycarbonyl;
R₂ is alkyl;
R₃ is a bond or methylene; and

R₄ is aryl or aralkyl; or
R₂ and R₄-R₃ are joined to form, together with the methine to which they are attached, a cyclic or polycyclic group;
and pharmaceutically acceptable salts thereof.

In certain preferred embodiments, the deprenyl is represented by the following general formula (Formula 6): 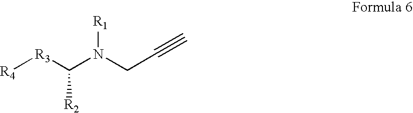
in which
R₁ is hydrogen, alkyl, alkenyl, alkynyl, aralkyl, alkylcarbonyl, arylcarbonyl, alkoxycarbonyl, or aryloxycarbonyl;
R₂ is alkyl;
R₃ is a bond or methylene; and
R₄ is aryl or aralkyl; or
R₂ and R₄-R₃ are joined to form, together with the methine to which they are attached, a cyclic or polycyclic group;
and pharmaceutically acceptable salts thereof.

In certain preferred embodiments, the deprenyl compound is represented by the following general formula (Formula 7): 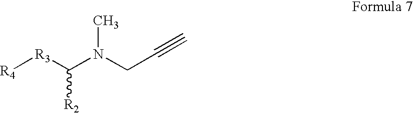
in which
R₂ is hydrogen or alkyl;
R₃ is a bond or methylene; and
R₄ is aryl or aralkyl; or
R₂ and R₄-R₃ are joined to form, together with the methine to which they are attached, a cyclic or polycyclic group; and

R₅ is alkylene, alkenylene, alkynylene and alkoxylene;
and pharmaceutically acceptable salts thereof.

In certain preferred embodiments, the deprenyl is represented by the following general formula (Formula 8): 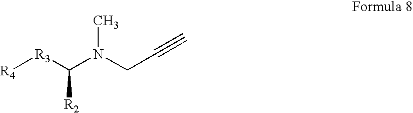
in which
R₁ is hydrogen, alkyl, alkenyl, alkynyl, aralkyl, alkylcarbonyl, arylcarbonyl, alkoxycarbonyl, or aryloxycarbonyl;
R₂ is alkyl;
R₃ is a bond or methylene; and
R₄ is aryl or aralkyl; or
R₂ and R₄-R₃ are joined to form, together with the methine to which they are attached, a cyclic or polycyclic group;
and pharmaceutically acceptable salts thereof.

In certain preferred embodiments, the deprenyl compound is represented by the following general formula (Formula 9): 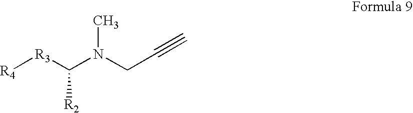
in which
R₁ is hydrogen, alkyl, alkenyl, alkynyl, aralkyl, alkylcarbonyl, arylcarbonyl, alkoxycarbonyl, or aryloxycarbonyl;
R₂ is alkyl;
R₃ is a bond or methylene; and
R₄ is aryl or aralkyl; or
R₂ and R₄-R₃ are joined to form, together with the methine to which they are attached, a cyclic or polycyciic group;
and pharmaceutically acceptable salts thereof.

In certain preferred embodiments, the deprenyl compound is represented by the following general formula (Formula 10): 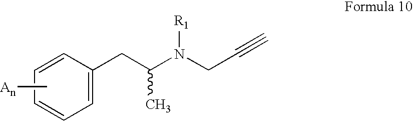
in which
R₁ is hydrogen, alkyl, alkenyl, alkynyl, aralkyl, alkylcarbonyl, arylcarbonyl, alkoxycarbonyl, or aryloxycarbonyl;
A is a substituent independently selected for each occurence from the group consisting of halogen, hydroxyl, alkyl, alkoxyl, cyano, nitro, amino, carboxyl, —CF₃, or azido;
n is 0 or an integer from 1 to 5;
and pharmaceutically acceptable salts thereof.

In certain preferred embodiments, the deprenyl is represented by the following general formula (Formula 11): 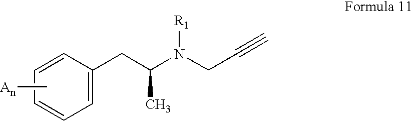
in which
R₁ is hydrogen, alkyl, alkenyl, alkynyl, aralkyl, alkylcarbonyl, arylcarbonyl, alkoxycarbonyl, or aryloxycarbonyl;
A is a substituent independently selected for each occurence from the group consisting of halogen, hydroxyl, alkyl, alkoxyl, cyano, nitro, amino, carboxyl, —CF₃, or azido;
n is 0 or an integer from 1 to 5;
and pharmaceutically acceptable salts thereof.

In certain preferred embodiments, the deprenyl is represented by the following general formula (Formula 12): 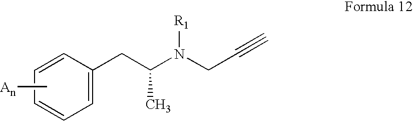
in which
R₁ is hydrogen, alkyl, alkenyl, alkynyl, aralkyl, alkylcarbonyl, arylcarbonyl, alkoxycarbonyl, or aryloxycarbonyl;
A is a substituent independently selected for each occurence from the group consisting of halogen, hydroxyl, alkyl, alkoxyl, cyano, nitro, amino, carboxyl, —CF₃, or azido;
n is 0 or an integer from 1 to 5;
and pharmaceutically acceptable salts thereof.

In a preferred embodiment, the deprenyl compound is deprenyl, more preferably (−)-deprenyl. In a particularly preferred embodiment, the deprenyl compound is desmethyldeprenyl, particularly (−)-desmethyldeprenyl. A large number of patients have been treated with (−)-deprenyl (e.g., for treatment of Parkinson'd disease) without evidence of significant deleterious side effects. Since (−)-deprenyl is largely metabolized to (−)-desmethyldeprenyl, it is unlikely that (−)-desmethyldeprenyl treatment will produce serious side effects.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 20 or fewer carbon atoms in its backbone (e.g., C₁-C₂₀ for straight chain, C₃-C₂₀ for branched chain), and more preferably 10 or fewer. Likewise, preferred cycloalkyls have from 4-10 carbon atoms in their ring structure, and more preferably have 5, 6 or 7 carbons in the ring structure. Unless the number of carbons is otherwise specified, “lower alkyl” as used herein means an alkyl group, as defined above, but having from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

Moreover, the term “alkyl” (or “lower alkyl”) as used throughout the specification and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, halogen, hydroxyl, carbonyl (such as carboxyl, ketones (including alkylcarbonyl and arylcarbonyl groups), and esters (including alkyloxycarbonyl and aryloxycarbonyl groups)), thiocarbonyl, acyloxy, alkoxyl, phosphoryl, phosphonate, phosphinate, amino, acylamino, amido, amidine, imino, cyano, nitro, azido, sulfhydryl, alkylthio, sulfate, sulfonate, sulfamoyl, sulfonamido, heterocyclyl, aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of aminos, azidos, iminos, amidos, phosphoryls (including phosphonates and phosphinates), sulfonyls (including sulfates, sulfonamidos, sulfamoyls and sulfonates), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The terms “alkenyl” and “alkynyl” refer to unsaturated aliphatic groups analogous in length and possible substitution to the alkyls described above, but that contain at least one double or triple bond respectively.

The term “aralkyl”, as used herein, refers to an alkyl or alkylenyl group substituted with at least one aryl group (e.g., an aromatic or heteroaromatic group). Exemplary aralkyls include benzyl (i.e., phenylmethyl), 2-naphthylethyl, 2-(2-pyridyl)propyl, 5-dibenzosuberyl, debenzo[b,f]oxepinylmethyl, and the like.

The term “alkylcarbonyl”, as used herein, refers to —C(O)-alkyl. Similarly, the term “arylcarbonyl” refers to —C(O)-aryl. The term “alkyloxycarbonyl”, as used herein, refers to the group —C(O)—O-alkyl, and the term “aryloxycarbonyl” refers to —C(O)—O-aryl. The term “acyloxy” refers to —O—C(O)—R₇, in which R₇ is alkyl, alkenyl, alkynyl, aryl, aralkyl or heterocyclyl.

The term “amino”, as used herein, refers to —N(R₈)(R₉), in which R₈ and R₉ are each independently hydrogen, alkyl, alkyenyl, alkynyl, aralkyl, aryl, or R₈ and R₉, together with the nitrogen atom to which they are attached, form a ring having 4-8 atoms. Thus, the term “amino”, as used herein, includes unsubstituted, monosubstituted (e.g., monoalkylamino or monoarylamino), and disubstituted (e.g., dialkylamino or alkylarylamino) amino groups. The term “amido” refers to —C(O)—N(R₈)(R₉), in which R₈ and R₉ are as defined above. The term “acylamino” refers to —N(R′₈)C(O)—R₇, in which R₇ is as defined above and R′₁₈ is alkyl.

As used herein, the term “nitro” means —NO₂; the term “halogen” designates —F, —Cl, —Br or —I; the term “sulfhydryl” means —SH; and the term “hydroxyl” means —OH.

The term “aryl” as used herein includes 5-, 6- and 7-membered aromatic groups that may include from zero to four heteroatoms in the ring, for example, phenyl, pyrrolyl, furyl, thiophenyl, imidazolyl, oxazole, thiazolyl, triazolyl, pyrazolyl, pyridyl, pyrazinyl, pyridazinyl and pyrimidinyl, and the like. Those aryl groups having heteroatoms in the ring structure may also be referred to as “aryl heterocycles” or “heteroaromatics”. The aromatic ring can be substituted at one or more ring positions with such substituents as described above for alkyls, for example, halogen, azide, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, sulfonamido, ketone, aldehyde, ester, a heterocyclyl, an aromatic or heteroaromatic moiety, —CF₃, —CN, or the like. Aryl groups can also be part of a polycyclic group. For example, aryl groups include fused aromatic moieties such as naphthyl, anthracenyl, quinolyl, indolyl, and the like.

The terms “heterocyclyl” or “heterocyclic group” refer to 4- to 10-membered ring structures, more preferably 4- to 7-membered rings, which ring structures include one to four heteroatoms. Heterocyclyl groups include, for example, pyrrolidine, oxolane, thiolane, imidazole, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycyclic group can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “heteroatom” as used herein means an atom of any element other than carbon or hydrogen. Preferred heteroatoms are nitrogen, oxygen, sulfur and phosphorus.

It will be noted that the structure of some of the compounds of this invention includes asymmetric carbon atoms. It is to be understood accordingly that the isomers arising from such asymmetry are included within the scope of this invention. Such isomers are obtained in substantially pure form by classical separation techniques and by sterically controlled synthesis.

The term “can be removed in vivo”, as used herein, refers to a group that can be cleaved in vivo, either enzymatically or non-enzymatically. For example, amides can be cleaved by amidases, and N-methyl amines can be cleaved by enzymatic oxidation. For example, when deprenyl is administered to a subject, it is believed, as described infra, that the methyl group can be removed in vivo to yield an active compound. As a further example, with reference to Formula I, when R₁ is alkylcarbonyl, the resulting amide group can be hydrolytically cleaved in vivo, enzymatically or non-enzymatically, to yield a deprenyl compound including a secondary amine (e.g., R₁ is converted to hydrogen in vivo). Other groups which can be removed in vivo are known (see, e.g., R. B. Silverman (1992) “The Organic Chemistry of Drug Design and Drug Action”, Academic Press, San Diego) and can be employed in compounds useful in the present invention.

II. Pharmaceutical Compositions

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject deprenyl compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The stability of deprenyl can be affected by the pH of the medium in which the deprenyl is formulated. For example, deprenyl is more stable at a pH in the range of about 3-5 than at a pH of about 7. Therefore, when formulating a deprenyl compound in a pharmaceutical composition, it is preferred that the deprenyl compound be maintained at a suitable pH. In preferred embodiments, a pharmaceutical composition of the invention has a pH in the range of about 3 to about 5, more preferably about 3 to about 4. Furthermore, ethyl alcohol is a preferred solvent for improving stability of deprenyl. Thus, in certain embodiments, alcoholic or aqueous alcoholic media are preferred for the pharmaceutical compositions of the invention.

As set out above, certain embodiments of the present deprenyl compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulfonate salts and the like (see, for example, Berge et al. (1977) “Pharmaceutical Salts”, J. Pharm. Sci. 66:1-19).

In other cases, the deprenyl compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the deprenyl compound which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent.

Methods of preparing these formulations or compositions include the step of bringing into association at least one deprenyl compound of the present invention with the carrier and, optionally, one or more accessory ingredients. In general, the formulations are prepared by uniformly and intimately bringing into association a deprenyl compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A deprenyl compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered deprenyl compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the deprenyl compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active deprenyl compound, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more deprenyl compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the deprenyl compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a deprenyl compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to a deprenyl compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the deprenyl compound in the proper medium. Absorption enhancers can also be used to increase the flux of the deprenyl compound across the skin. The rate of such flux can be controlled by either providing a rate controlling membrane or dispersing the deprenyl compound in a polymer matrix or gel. In a preferred embodiment, a deprenyl compound is administered by transdermal application, e.g., by use of a transdermal patch. Devices, including patches, which transdermally deliver a deprenyl compound by iontophoresis or other electrically-assisted methods can also be employed in the present invention, including, for example, the devices described in U.S. Pat. Nos. 4,708,716 and 5,372,579.

Ophthalmic formulations, eye ointments, powders, solutions, drops, sprays and the like, are also contemplated as being within the scope of this invention.

Pharmaceutical compositions of this invention suitable for parenteral administration comprise one or more deprenyl compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject deprenyl compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given alone or as a pharmaceutical composition containing, for example, 0.01 to 99.5% (more preferably, 0.1 to 90%) of active ingredient in combination with a pharmaceutically acceptable carrier.

The preparations of the present invention may be given orally, parenterally, topically, or rectally. They are of course given by forms suitable for each administration route. For example, they are administered in tablets or capsule form, by injection, inhalation, eye lotion, ointment, suppository, etc.; administration by injection, infusion or inhalation; topical by lotion or ointment; and rectal by suppositories. Injection (subcutaneous or intraperitoneal) or topical ophthalmic administration are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular deprenyl compound of the present invention employed, or the ester, salt or amide thereof, the route of administration, the time of administration, the rate of excretion of the particular compound being employed, the duration of the treatment, other drugs, compounds and/or materials used in combination with the particular deprenyl compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a deprenyl compound of the invention will be that amount of the compound which is the lowest dose effective to produce a therapeutic effect. Such an effective dose will generally depend upon the factors described above. Generally, intraperitoneal and subcutaneous doses of the compounds of this invention for a patient, when used for the indicated Schwann cell rescuing effects, will range from about 0.0001 to about 10 mg per kilogram of body weight per day, more preferably from about 0.001 mg/kg to about 1 mg/kg per day, still more preferably from about 0.01 mg to about 0.5 mg/kg/day. The deprenyl compound can be administered in a single dose, or the therapy can be administered over an extended period, either by use of a sustained-relaease or depot formulation as described herein, or by repreated administration over an extended period, e.g., over a period of one week, one month, six months, one yer, two years, five years, or ten years, or even longer if desired.

If desired, the effective daily dose of a deprenyl compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition). It will be understood that two or more deprenyl compounds can be administered in a single therapeutic composition.

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are well known to those skilled in the art.

In certain embodiments, it is preferable to administer deprenyl compounds by a route that minimizes metabolism to inhibitor compounds such as (−)-methamphetamine and (−)-amphetamine, while allowing metabolism to active compounds such as (−)-desmethyldeprenyl. Metabolism to an active compound can occur at the desired site of activity, e.g., in the target organ or area, e.g., the brain. Thus, prodrugs, which are metabolized to active compounds, are useful in the methods of the invention. It has been found that certain deprenyl compounds have greater therapeutic efficacy (e.g., are effective at lower doses) when administered so as to decrease or prevent the “first-pass” effect. Accordingly, intraperitoneal or especially subcutaneous injection are preferred routes of administration. In preferred embodiments, a deprenyl compound is administered in divided doses. For example, a deprenyl compound can be administered by frequent (e.g., pulsed) injections, or by a controlled infusion, which can be constant or programmably varied as described above. In preferred embodiments in which a deprenyl compound is administered orally, the deprenyl compound can be formulated to reduce the amount of hepatic metabolism after oral administration and thereby improve the therapeutic efficacy.

In certain embodiments, the deprenyl compounds of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties”), thus providing targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); gp120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In a preferred embodiment, the therapeutic compounds of the invention are formulated in liposomes; in a more preferred embodiment, the liposomes include a targeting moiety.

The following invention is further illustrated by the following example, which should in no way be construed as being further limiting. The contents of all references, pending patent applications and published patent applications, cited throughout this application are hereby incorporated by reference.

The following non-limiting examples are illustrative of the present invention:

Experimental Details

### EXAMPLE 1

A photoaffinity-labelled derivative of a deprenyl analog was prepared and (−)-desmethyldeprenyl was “tagged” with the fluorescent group BODIPY. The addition of the photoaffinity analog to PC 12 cells and extraction of protein from the cells, followed by SDS polyacrylamide gel electrophoresis (PAGE) and subsequent Western blot analysis, showed that one of the proteins which bound the analog was GAPDH. The tagged compound was incubated with PC 12 cells, and the intracellular location of the tagged compound was examined using immunofluorescence and confocal microscopy. It was found that tagged DMD co-localized with GADPH. In addition, binding of DMD to GAPDH was blocked by incubating GAPDH with a sheep polyclonal antibody to GAPDH. It was determined that the polyclonal antibody blocks access to the channel of GAPDH tetrameric. Gel filtration of GAPDH incubated with DMD showed the presence of a lower molecular weight peak; this was determined to be a dimeric form of GAPDH. Thus, DMD can associate with GAPDH and disrupt the association of GAPDH monomers, thus inducing the conversion of GAPDH from the tetrameric form to the dimeric form.

Computer modelling suggested that the most probable binding site for DMD on GAPDH is in or near the “channel” formed by the four associated monomeric units of GAPDH. Moreover, molecular modelling indicated that binding of DMD to this binding site could result in a change in the conformation of GAPDH.

### EXAMPLE 2

To demonstrate that deprenyl compounds can prevent cell death due to viral infection, in vitro experiments were performed. It is known that in some viral diseases or conditions, including LCM-mediated choriomeningitis, AIDS dementia, and Boma disease in livestock, pathogenesis is largely mediated by the immune response to viral infection, and in particular by CTL, rather than by a direct effect of the virus itself. To model this situation, CTL-mediated killing of several cell lines which expressed a viral protein (Simian Immunodeficiency virus envelope protein (“SIV env”)) was measured in the presence and absence of deprenyl compounds. Cell killing was measured by studying release of ⁵¹Cr from the cells.

The experiments used four different cell lines, each expressing SIV env: huBLCL/env, rh BLCL/env, ClR/env and Hela T4/env cells. huBLCL/env is a human Epstein Barr virus (EBV) derived cell line, and rh BLCL/env is from rhesus monkey. C1R line is a human B cell line that is deficient in expressing MHC class I and class II molecules; it was used so that we could observe MHC unrestricted killing. The percent lysis is calculated as (100)×(⁵¹Cr released from the sample well-spontaneous release)/(total release-spontaneous release). To block CTL-mediated lysis, compounds were added at the beginning of each 5 hour assay. Media was added as a control and other compounds were added at 1.0 nM concentration. N-acetyl cysteine (NAC), and (−)-desmethyldeprenyl have been shown to have anti-apoptotic activities.

Percent Specific Lysis

| Target Cells | Control | NAC | Desm. |
|---|---|---|---|
| hu BLCL/env | 14.08 | 13.41 | −1.85 |
| rh BLCL/env | 11.29 | 6.32 | 4.34 |
| ClR/env | 38.97 | 29.12 | 6.98 |
| Hela T4/env | 49.16 | 30.74 | 30.65 |

The results suggest that (−)-desmethyldeprenyl can reduce cell death, in particular CTL-mediated cell death, due to viral infection in vitro. (−)-Desmethyldeprenyl reduced cell killing in all four cell lines and was the more consistent than NAC in blocking chromium release or “Specific Lysis.” NAC was effective in reducing lysis in the rh BLCL/env, C1R/env and Hela T4/env cells, but not in the huBLCL/env cells.

### REFERENCES

Anderson L E, Li D, Prakash N, Stevens F J (1995) Identification of potential redox-sensitive cysteines in cytosolic forms of fructosebisphosphatase and glyceraldehyde-3-phosphate dehydrogenase. Planta, 196, 118-124.
Bereta J, Bereta M (1995) Stimulation of glyceraldehyde-3-phosphate dehydrogenase mRNA levels by endogenous nitric oxide in cytokine-activated endothelium. Biochemical and Biophysical Research Communications, 217, 363-369.
Brune B, Lapetina E G (1996) Nitric oxide-induced covalent modification of glycolytic enzyme glyceraldehyde-3phosphate dehydrogenase. In: Nitric Oxide, Pt B. Edited by L. Packer. 525 B Street/Suite 1900/San Diego/CA 92101-4495: Academic Press Inc, pp. 400-407.
Burke J R, Enghild J J, Martin M E, Jou Y S, Myers R M, Roses A D et al (1996) Huntington and DRPLA proteins selectively interact with the enzyme GAPDH. Nature Medicine, 2, 347-350.
Chalmers-Redman R M E, Fraser A D, Ju W Y H, Wadia J, Tatton N A, Tatton W G (1996) Mechanisms of nerve cell death: apoptosis or necrosis after cerebral ischemia. In: International Review Of Neurobiology Neuroprotective Agents and Cerebral Ischemia, Volume 40. Edited by A. R. Green and A. J. Cross. London:
   Academic Press, pp. 1-26.
Del Llano A M, Layergne J A (1994) Why do cells die in HIV infection? Potential mechanisms inducing programmed cell death/apoptosis. P R Health Sci J, 13, 79-124.
Doucas V, et. al. (1996) Adenovirus replication is coupled with the dynamic properties of the PML nuclear structure. Adenovirus replication is coupled with the dynamic properties of the PML nuclear structure. Genes and Devt., 10, 196-207.
Dragunow M, Faull R L, Lawlor P, Beilharz E J, Singleton K, Walker E B et al. (1995) In situ evidence for DNA fragmentation in Huntington's disease striatum and Alzheimer's disease temporal lobes. Neuroreport, 6, 1053-7.
Erecinska M, Nelson D, Vanderkooi J M (1995) Effects of NO-generating compounds on synaptosomal energy metabolism. Journal of Neurochemistry, 65, 2699-2705.
Ishitani R, Chuang D M (1996) Glyceraldehyde-3-phosphate dehydrogenase antisense oligodeoxynucleotides protect against cytosine arabinonucleoside-induced apoptosis in cultured cerebellar neurons. Proceedings of the National Academy of Sciences of the United States of America, 93, 9937-9941.
Ishitani R, Kimura M, Sunaga K, Katsube N, Tanaka M, Chuang D M (1996a) An antisense oligodeoxynucleotide to glyceraldehyde-3-phosphate dehydrogenase blocks age-induced apoptosis of mature cerebrocortical neurons in culture. Journal of Pharmacology and Experimental Therapeutics, 278, 447-454.
Ishitani R, Sunaga K, Hirano A, Saunders P, Katsube N, Chuang D M (1996b) Evidence that glyceraldehyde-3phosphate dehydrogenase is involved in age-induced apoptosis in mature cerebellar neurons in culture. Journal of Neurochemistry, 66, 928-935.
Ishov A M, Maul G G (1996) The periphery of Nuclear Domain 10 (NDIO) as site of DNA virus deposition. J. Cell Biol., 134, 815-826.
Ito Y, Pagano P J, Tornheim K, Brecher P, Cohen R A (1996) Oxidative stress increases glyceraldehyde-3phosphate dehydrogenase mRNA levels in isolated rabbit aorta. American Journal of Physiology Heart and Circulatory Physiology, 39, H81-H87.
Kalams S A, Walker B D (1992) Cytotoxic T Lymphocytes and HIV-I Related Neurologic Disorders. Current Opinions in Microbiology and Immunology., 202.
Kelly C, VanDriel R, Wilkinson G W G (1995) Disruption of PML-associated nuclear bodies during human cytomegalovirus infection. Journal of General Virology, 76, 2887-2893.
Mateo R B, Reichner J S, Mastrofrancesco B, KraftStolar D, Albina J E (1995) Impact of nitric oxide on macrophage glucose metabolism and glyceraldehyde-3-phosphate dehydrogenase activity. American Journal of Physiology Cell Physiology, 37, C669-C675.
Maul G G, Guldner H H, Spivack J G (1993) Modification of discrete nuclear domains induced by herpes simplex virus type I immediate early gene 1 product (ICPO). J. Gen. Virol., 74.
Morimoto K, et al. (1996) Intrinsic responses to Borna disease virus infection of the central nervous system. Proc. Natl. Acad. Sci., 93, 13345-13350.
Muller W E, Schroder H C, Ushijima H, Dapper J, Bormann J (1992) Gpl20 of Hiv-1 induces apoptosis in rat cortical cell cultures: prevention by memantine. Eur J Pharmacol, 226, 209-14.
Nagy E, Rigby W F C (1995) Glyceraldehyde-3-phosphate dehydrogenase selectively binds AU-rich RNA in the NAD(+)-binding region (Rossmann fold). Journal of Biological Chemistry, 270, 2755-2763.
Padgett C M, Whorton A R (1997) Glutathione redox cycle regulates nitric oxide-mediated glyceraldehyde-3phosphate dehydrogenase inhibition. American Journal of Physiology Cell Physiology, 41, C99-C 108.
Portera-Cailliau C, Hedreen J C, Price D L, Koliatsos V E (1995) Evidence for apoptotic cell death in Huntington disease and excitotoxic animal models. J. Neurosci., 15, 3775-3787.
Puvion-Dutillenl F, et al. (1995) Adenovirus infection induces rearrangements in the intranuclear distribution of the nuclear body associated PML protein. Expt. Cell Res., 218, 9-16.
Salvato M, Rai S K (1996) Arenaviruses. In: Topley and Wilson's Medical Microbiology, Volume 13: Arnold Publishers, London.
Schultz D E, Hardin C C, Lemon S M (1996) Specific interaction of glyceraldehyde 3-phosphate dehydrogenase with the 5′-nontranslated RNA of hepatitis A virus. Journal of Biological Chemistry, 271, 14134-14142.
Sirover M A (1996) Emerging new functions of the glycolytic protein, glyceraldehyde-3-phosphate dehydrogenase, in mammalian cells. Life Sciences, 58, 2271-2277.
Sobbe M, et al. (1997) Induction of degenerative brain lesions after adoptive transfer of brain lymphocytes from Borna disease virus infected rats: presence of CD8+ T cells and perforin mRNA. J. Virol., 71, 2400-2407.
Sunaga K, Takahashi H, Chuang D M, Ishitani R (1995) Glyceraldehyde-3-phosphate dehydrogenase is overexpressed during apoptotic death of neuronal cultures and is recognized by a monoclonal antibody against amyloid plaques from Alzheimer's brain. Neuroscience Letters, 200, 133-136.
Szekely Lea (1996) The Epstein-Barr virus encoded nuclear antigen EBNA-5 accumulates in PML containing bodies. J. Virol., 70, 2562-2568.
Tamaoka A, Endoh R, Shoji S, Takahashi H, Hirokawa K, Teplow D B et al. (1996) Antibodies to amyloid beta protein (A beta) crossreact with glyceraldehyde-3-phosphate dehydrogenase (GAPDH). Neurobiology of Aging, 17, 405-414.
Tatton W G, Chalmers-Redman R M E (1996) Modulation of gene expression rather than monoamine oxidase inhibition: (−)-deprenyl-related compounds in controlling neurodegeneration. Neurology, 47, S 171-S 183.
Tatton W G, Ju W J H, Wadia J, Tatton N A (1996) Reduction of neuronal apoptosis by small molecules: promise for new approaches to neurological therapy. In: Neuroprotection and Neurodegeneration. Edited by W. Olanow, M. Youdim and P. Jenner. New York: Academic Press Ltd., pp. 209-229.
Thomas L B, Gates D J, Richfield E K, T F O B, Schweitzer J B, Steindler D A (1995) DNA end labeling (TUNEL) in Huntington's disease and other neuropathological conditions. Exp Neurol, 133, 265-72.
Volker K W, Reinitz C A, Knull H R (1995) Glycolytic enzymes and assembly of microtubule networks. Comparative Biochemistry and Physiology B Biochemistry & Molecular Biology, 112, 503-514.

1. A method of treating a viral infection, comprising administering to a subject in need thereof a therapeutically effective amount of a deprenyl compound, such that treatment of the viral infection occurs.

2. The method of claim 1, wherein the viral infection is caused by an RNA virus.

3. The method of claim 2, wherein said RNA virus is selected from the group consisting of HIV, Herpes Simplex-1 virus, hepatitis A virus, Epstein-Barr virus, SV-40 virus, cytomeglavirus and adenovirus-5.

4. The method of claim 1, wherein the deprenyl compound is represented by the formula: 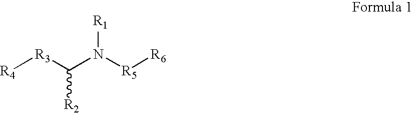
in which
R₁ is hydrogen, alkyl, alkenyl, alkynyl, aralkyl, alkylcarbonyl, arylcarbonyl, alkoxycarbonyl, or aryloxycarbonyl;
R₂ is hydrogen or alkyl;
R₃ is a single bond, alkylene, or —(CH₂)ₙ—X—(CH₂)ₘ;
in which X is O, S, or N-methyl; m is 1 or 2; and n is 0,1, or 2;
R₄ is alkyl, alkenyl, alkynyl, heterocyclyl, aryl or aralkyl; and
R₅ is alkylene, alkenylene, alkynylene and alkoxylene; and
R₆ is C₃-C₆ cycloalkyl or
—C≡CH; or
R₂ and R₄-R₃ are joined to form, together with the methine to which they are attached, a cyclic or polycyclic group;
and pharmaceutically acceptable salts thereof.

5. The method of claim 1, wherein the deprenyl compound is (−)-desmethyldeprenyl.

6. The method of claim 1, wherein the deprenyl compound is administered to the subject by transdermal administration.

7. The method of claim 1, wherein the deprenyl compound is administered in a pharmaceutically acceptable carrier.

8. The method of claim 1, wherein the subject is a human.

9. A method of inhibiting replication of a virus in a virus-infected cell, comprising contacting the virus-infected cell with an effective amount of a deprenyl compound, such that the affinity of GAPDH for viral RNA is decreased and viral replication in the virus-infected cell is inhibited.

10. The method of claim 9, wherein the virus is selected from the group consisting of HIV, Herpes Simplex-1 virus, hepatitis A virus, Epstein-Barr virus, SV-40 virus, cytomeglavirus and adenovirus-5.

11. The method of claim 9, wherein the virus-infected cell is a cell in cell culture.

12. The method of claim 9, wherein the deprenyl compound is represented by the formula: 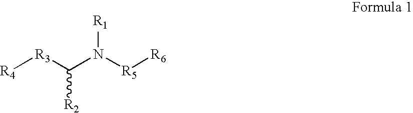
in which
R₁ is hydrogen, alkyl, alkenyl, alkynyl, aralkyl, alkylcarbonyl, arylcarbonyl, alkoxycarbonyl, or aryloxycarbonyl;
R₂ is hydrogen or alkyl;
R₃ is a single bond, alkylene, or —(CH₂)ₙ—X—(CH₂)ₘ;
in which X is O, S, or N-methyl; m is 1 or 2; and n is 0,1, or 2;
R₄ is alkyl, alkenyl, alkynyl, heterocyclyl, aryl or aralkyl; and
R₅ is alkylene, alkenylene, alkynylene and alkoxylene; and
R₆ is C₃-C₆ cycloalkyl or
—C≡CH; or
R₂ and R₄-R₃ are joined to form, together with the methine to which they are attached, a cyclic or polycyclic group;
and pharmaceutically acceptable salts thereof.

13. The method of claim 12, wherein the deprenyl compound is (−)-desmethyldeprenyl.

14. A method for decreasing the affinity of GAPDH for viral RNA, the method comprising contacting GAPDH with a deprenyl compound, such that the affinity of GAPDH for viral RNA is decreased.

15. The method of claim 14, wherein the deprenyl compound associates with GAPDH such that the conformation of GAPDH is altered.

16. The method of claim 14, wherein the deprenyl compound is represented by the formula: 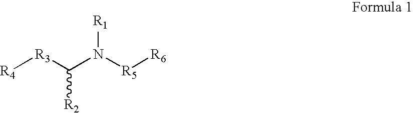
in which
R₁ is hydrogen, alkyl, alkenyl, alkynyl, aralkyl, alkylcarbonyl, arylcarbonyl, alkoxycarbonyl, or aryloxycarbonyl;
R₂ is hydrogen or alkyl;
R₃ is a single bond, alkylene, or —(CH₂)ₙ—X—(CH₂)ₘ;
in which X is O, S, or N-methyl; m is 1 or 2; and n is 0,1, or 2;
R₄ is alkyl, alkenyl, alkynyl, heterocyclyl, aryl or aralkyl; and
R₅ is alkylene, alkenylene, alkynylene and alkoxylene; and
R₆ is C₃-C₆ cycloalkyl or
—C≡CH; or
R₂ and R₄-R₃ are joined to form, together with the methine to which they are attached, a cyclic or polycyclic group;
and pharmaceutically acceptable salts thereof.

17. The method of claim 16, wherein the deprenyl compound is (−)-desmethyldeprenyl.

18. A method for inhibiting replication of a virus in a virus-infected cell, comprising inhibiting colocalization of GAPDH with PML such that replication of the virus in the virus-infected cell is inhibited.

19. The method of claim 18, wherein the colocalization of GAPDH with PML is inhibited by contacting GAPDH with a depreneyl compound.

20. A method for inhibiting tissue damage due to viral infection, comprising administering to a subject in need thereof an effective amount of a deprenyl compound such that prevention of tissue damage due to viral infection occurs.

21. The method of claim 20, wherein said viral infection is selected from the group consisting of HIV, Herpes Simplex-1 virus, hepatitis A virus, Epstein-Barr virus, SV-40 virus, cytomeglavirus and adenovirus-5.

22. The method of claim 20, wherein the deprenyl compound is represented by the formula: 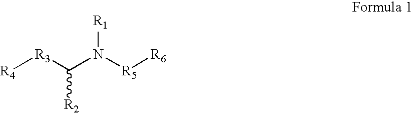
in which
R₁ is hydrogen, alkyl, alkenyl, alkynyl, aralkyl, alkylcarbonyl, arylcarbonyl, alkoxycarbonyl, or aryloxycarbonyl;
R₂ is hydrogen or alkyl;
R₃ is a single bond, alkylene, or —(CH₂)ₙ—X—(CH₂)ₘ;
in which X is O, S, or N-methyl; m is 1 or 2; and n is 0,1, or 2;
R₄ is alkyl, alkenyl, alkynyl, heterocyclyl, aryl or aralkyl; and
R₅ is alkylene, alkenylene, alkynylene and alkoxylene; and
R₆ is C₃-C₆ cycloalkyl or
—C≡CH; or
R₂ and R₄-R₃ are joined to form, together with the methine to which they are attached, a cyclic or polycyclic group;
and pharmaceutically acceptable salts thereof.

23. The method of claim 22, wherein the deprenyl compound is (−)-desmethyldeprenyl.
